(19) United States
(12) Patent Application Publication
(10) Pub. No.: US 2012/0021060 A1
(43) Pub. Date: Jan. 26, 2012

(54) Honey Compositions and Uses Thereof

(76) Inventors: Amin Ramzanali Javer, Vancouver (CA); Ezra Kwok, Vancouver (CA)

(21) Appl. No.: 12/841,040

(22) Filed: Jul. 21, 2010

(51) Int. Cl. A61K 35/64; A61P 11/00; A61P 31/00

(52) U.S. Cl. 424/537

(57) ABSTRACT

A composition for treating or alleviating the symptoms of sinusitis comprising Manuka honey at a concentration of from about 0.5% v/v to about 7% v/v in an aqueous pH-balanced solution is provided. In one embodiment, the concentration of Manuka honey is from about 3% v/v to about 6% v/v. In another embodiment, the concentration of Manuka honey is from about 4% v/v to about 5% v/v. A method of treating or alleviating the symptoms of sinusitis in a patient in need of such treatment, where the method comprises intranasally administrating the above composition to such patient in order to treat the sinusitis, is provided. A further method of improving olfactory function in a patient in need of treatment is provided, where the method comprises intranasally administering the above composition to such patient in order to improve olfactory function.

### TECHNICAL FIELD

The present invention relates to honey compositions and their use to treat sinusitis and improve olfactory function.

### BACKGROUND

Sinusitis is an inflammation of the mucous membrane of any sinus, especially the paranasal sinuses, which may be due to infection from a virus, bacteria or fungus, or the result of an allergic or autoimmune reaction. Sinusitis is a common condition with between 24 and 31 million cases occurring in the United States annually.

One form of sinusitis is chronic rhinosinusitis (CRS). CRS is a common and often debilitating form of sinusitis. It affects over 40 million people worldwide, with more than 200,000 requiring some form of medical intervention (Loehrl T. A., “Autonomic function and dysfunction of the nose and sinuses”, Otolaryngol Clin North Am, 38(6): 1155-61, 2005). CRS typically lasts for a minimum of about 12 weeks and may often persist for longer periods of time. CRS may be caused by many different diseases that share chronic inflammation of the sinuses as a common symptom, for example cystic fibrosis (“CF”). CRS is highly prevalent among patients with CF and causes many complications for these patients. Potential problems include high risk of lower respiratory tract infections, sinus pain and pressure, nasal obstruction, hyposmia or anosmia, and more importantly increased risk of pneumonia following lung transplantation. Common treatments for patients diagnosed with CRS include functional endoscopic sinus surgery (FESS) and adjunctive medical therapies.

Allergic fungal rhinosinusitis (“AFRS”) is another form of sinusitis. AFRS is a chronic condition, and often a life debilitating disease. The nature of its etiology and pathogenesis is not well understood and a curative treatment for AFRS has not been identified. Patients with AFRS often undergo surgery; however, some patients are refractory to post-operative medical treatments and continue to suffer from inflammation of the sinuses.

To date, there has been minimal research in finding alternative therapeutic agents for sinusitis with increased efficacy. The effectiveness of a possible alternative therapeutic agent is dependent on its ability to reduce inflammation, and to eradicate the underlying pathogens including bacteria and fungus.

Honey has been used for the treatment of many disorders because of its anti-microbial and anti-inflammatory properties. Various forms of honey have been used to treat non-healing wounds, such as diabetic foot ulcers, first and second degree burns, donor sites, traumatic and surgical wounds, and to treat infections and other ailments. Honey's ability to act as an antimicrobial is due to the acidic pH and osmotic effect of the honey as well as several key ingredients, including, for example, hydrogen peroxide produced by honey glucose oxidase and flavonoid pinocembrin. Moreover, the glucose in honey provides energy to vital cells, like phagocytes, which are in need of energy production in areas where oxygen supply is often deficient.

Monofloral honey, such as Manuka honey, is produced from bees who feed on the flowers of the Manuka bush, also known as the “Tea Tree”. Manuka honey is known to have have anti-bacterial properties.

### SUMMARY OF THE INVENTION

We have unexpectedly discovered that low concentrations of Manuka honey, in a pH-balanced aqueous carrier can be used to significantly decrease the symptoms associated with sinusitis.

One exemplary embodiment relates to a composition for treating or alleviating the symptoms of sinusitis comprising Manuka honey at a concentration of from about 0.5% v/v to about 7% v/v in an aqueous pH-balanced solution.

According to one aspect, there is provided a composition comprising Manuka honey at a concentration of from about 3% v/v to about 6% v/v.

According to another aspect, there is provided a composition comprising Manuka honey at a concentration of from about 4% v/v to about 5% v/v.

Another exemplary embodiment is directed to a method for treating or alleviating the symptoms of sinusitis in a patient, where the method comprises the intranasal administration of a composition comprising Manuka honey at a concentration of from about 0.5% v/v to about 7% v/v in an aqueous pH-balanced solution to a patient requiring treatment in order to treat the sinusitis in the patient.

A further embodiment relates to methods for improving olfactory function in a patient requiring such improvement. The method comprises the intranasal administration of a composition comprising Manuka honey at a concentration of from about 0.5% v/v to about 7% v/v in an aqueous pH-balanced solution to the patient in order to improve the olfactory function of the patient.

### BRIEF DESCRIPTION OF THE DRAWINGS

The following figures set forth embodiments in which like reference numerals denote like parts. Embodiments are illustrated by way of example and not by way of limitation in the accompanying figures.

FIG. 1 is a photograph of the Sniffin' Sticks used to conduct olfactory tests;

FIG. 2a is a graph showing the mean overall SNOT-22 scores before and after 30 days of sinus irrigation with honey;

FIG. 2b is a graph comparing average pre- and post-honey treatment SNOT-22 scores for nasal obstruction, facial pain and loss of smell or taste;

FIG. 3 is a graph showing a comparison of mean endoscopic staging scores before and after 30 days of sinus irrigation with honey;

FIG. 4a is a graph showing a comparison of the mean total olfactory score (TDI) assessed by the Sniffin' Sticks test before and after 30 days of sinus irrigation with honey; and

FIG. 4b is a graph comparing average pre- and post-honey treatment olfactory scores for individual olfactory component: Threshold (T), Discrimination (D) and Identification (I).

### DETAILED DESCRIPTION

### Definitions

For purposes of this description, the following terms as defined in this section apply, unless the context of the word indicates a different meaning.

As used herein the term “sinus” is intended to mean all sinuses, i.e., the maxillary, ethmoid, frontal, and sphenoidal sinuses.

As used herein, the term “subject” is intended to mean mammalian subjects, typically humans. Mammals also include, but are not limited to, primates, farm animals, sport animals, cats, dogs, rabbits, mice, and rats. The terms “patient” or “subject” are used herein interchangeably with “mammal.”

As used herein, the term “treat”, “treating”, or “treatment” is intended to mean the resolution, reduction, or prevention of sinusitis or the sequelae of sinusitis.

As used herein, the term “nasal mucosa” is intended to mean the lining of the vestibule of the nose, where vascularized, and extending interiorly to the boundaries of the oropharynx and sinuses.

As used herein, the term “aqueous” is intended to mean a solution formed in water, but may contain lesser amounts of other co-solvents, such as salt and baking soda.

As used herein, the term “pharmaceutically acceptable” is intended to mean a composition which when administered to a human or a mammal by the indicated route of administration, provokes no adverse reaction which is disproportionate to the benefit gained by administration of the composition.

As used herein, the term “intranasal administration” or “intranasal delivery” is intended to mean delivery of the honey compositions, as described herein, primarily via the mucosa of the nasal cavity. This includes the superior, middle and inferior nasal turbinates and the nasal pharynx. Note that the olfactory region is concentrated in the superior (upper ⅓) of the nasal turbinates. Cilial action pushes material back toward the oropharynx, so material deposited in the nasal vestibule encounters the nasal mucosa before entering the throat.

I. Honey Compositions

We have discovered to our surprise that low concentrations of honey in aqueous saline solutions, when administered intranasally as a spray to treat sinusitis, can significantly alleviate the symptoms of sinusitis in human patients without causing nasal discomfort during administration. Moreover, we have discovered that the sprays can be administered to achieve beneficial results in human patients who are suffering from post-operative sinusitis. In one particular example, patients suffering from sinusitis secondary to cystic fibrosis who have undergone functional endoscopic sinus surgery have demonstrated unexpected olfactory improvement after nasal administration of the honey compositions. Such improvement includes the ability to discriminate and identify different odours.

Honey compositions include types of honey having strong antibacterial and antifungal properties. For example, the nectar from the flowers of the Leptospermum plant genus (also known as Manuka or the Tea Tree) is used in the making of Leptospermum honey also known as Manuka honey.

Thus, there is provided a composition for treating or alleviating the symptoms of sinusitis, the composition comprising: Manuka honey at a concentration of from about 0.5% v/v to about 7% v/v in an aqueous, pH-balanced solution.

The concentration of the Manuka honey is typically in the range of from about 1% v/v to about 7% v/v. In one embodiment, the Manuka honey is present at a concentration from about 3% v/v to about 6% v/v. In another embodiment, the concentration of the Manuka honey is from about 4% v/v to about 5% v/v.

In a further alternative embodiment, the composition consists of 0.5% v/v Manuka honey and 99.5% v/v of the aqueous pH-balanced solution. In another specific example, the composition consists of 5% v/v Manuka honey and 95% of the aqueous pH-balanced solution. In another specific example, the composition consists of 7% v/v Manuka honey and 93% of the aqueous pH-balanced solution.

In another alternative embodiment, the composition consists essentially of 0.5% v/v Manuka honey and 99.5% of the aqueous pH-balanced solution. In yet another specific example, the composition consists essentially of 5% v/v Manuka honey and 95% of the aqueous pH-balanced solution. In still another specific example, the composition consists essentially of 7% v/v Manuka honey and 93% of the aqueous pH-balanced solution.

The compositions described herein are aqueous pH-balanced solutions, which are compatible for human use. In the examples described herein, the aqueous pH-balanced solution includes a mixture of sodium chloride and sodium bicarbonate. The solution has the required osmolality for human use and is pharmaceutically acceptable for use as a nasal spray and has a final pH that is about pH 7.0.

Additional excipients may be used in the composition. These may include but are not limited to benzalkonium chloride, benzyl alcohol, phenylethyl alcohol, polyethylene glycol, glycerin, water, sodium hydroxide, aloe vera, camphor, boric acid, ascorbic acid, and citric acid.

II. Utilities

The compositions of this discovery are useful to treat, or alleviate the symptoms of sinusitis in human patients or affect olfactory function and comprise intranasally administering the composition to the patient in need thereof to treat the sinusitis. Specifically, the compositions are useful to treat chronic rhinosinusitis or the sinusitis is after functional endoscopic sinus surgery. Also, allergic fungal rhinosinusitis is also contemplated as being treatable using the honey compositions described herein. Further, the compositions are useful to improve general olfactory function.

Typically, the composition is administered every 24 hours. In one example, the composition is administered every 24 hours for 30 days.

Typically, the patient irrigates each of their nostrils with about 120 mL of the prepared honey solution, every 24 hours for 30 days

Several delivery mechanisms are known to those skilled in the art and include, for example, actuator dispensers commonly used for nasal solutions and gels. It is possible to use such dispensers to administer multiple, single-dose, and metered doses. Such dispensers may be child resistant, and disposable, and may be sold in kit format.

The above-described honey compositions can be administered to the nasal mucosa in flush form, liquid stream form, drop form or in spray form. However, the typical mode of administration is in flush form or liquid stream form. An example of suitable sinus rinse delivery mechanisms include the NeilMed® Sinus Rinse Bottle produced by NeilMed® Pharmaceuticals, Inc., California, a medical syringe of about 20 to 60 ml in size, and other squeeze bottle irrigation devices. A person skilled in the art would understand that other delivery mechanisms may also be used for delivery of the honey compositions.

### EXAMPLES

### Example 1

### Preparation of a Honey-Saline Solution

A honey-saline solution was prepared by dissolving Wedderspoon® Manuka honey (from Wedderspoon Organic®) in a pH-balanced sodium chloride and sodium bicarbonate mixture. The sodium chloride and sodium bicarbonate mixture was made by dissolving 1 Sinus Rinse™ packet in 240 mL of distilled water. The Manuka honey and the sodium chloride and sodium bicarbonate mixture were combined in the various volume to volume ratios as shown in Table 1 to prepare honey-saline solutions with different concentrations of honey:

TABLE 1

| Solution | Honey (cc) | Saline Mixture (ml) |
|---|---|---|
| 1 | 2.5 | 240 |
| 2 | 5 | 240 |
| 3 | 7.5 | 240 |
| 4 | 10 | 240 |
| 5 | 12.5 | 240 |
| 6 | 15 | 240 |
| 7 | 18 | 240 |

### Example 2

### Improving Sinusitis Symptoms in Cystic Fibrosis Patients Who Continue to Suffer from CRS Following Surgery

Ten patients with cystic fibrosis (“CF”) who were diagnosed with chronic rhinosinusitis and had undergone functional endoscopic sinus surgery (“FESS”) at least three months prior to the study were enrolled in a prospective study. The ten patients were a mean age of 31.9 years (range 21-48 years) and six of the patients were males (60%) and four of the patients were females (40%). All study subjects provided informed consents.

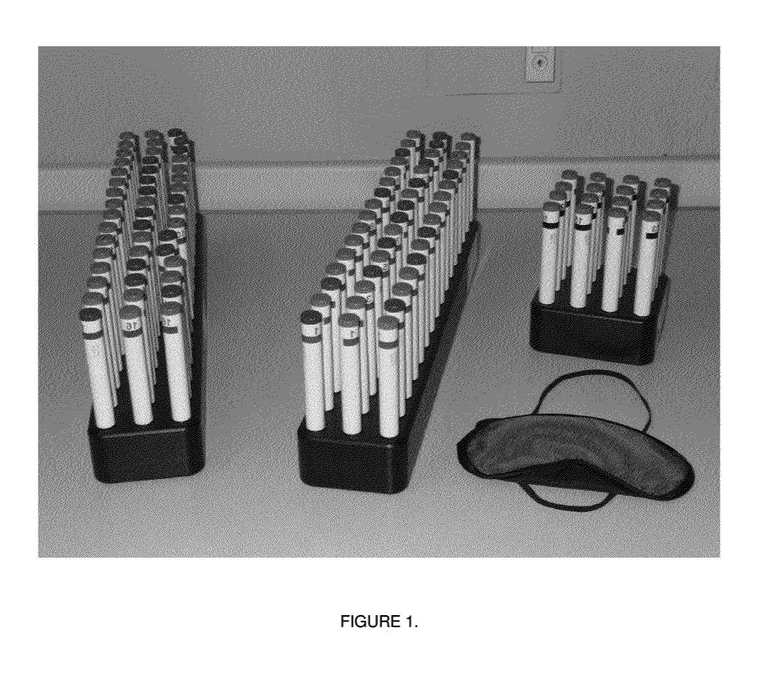

STEP 1: Olfactory Test & Health-Related Questionnaire. On the first day of the study, patients completed a Sniffin' Sticks olfactory test (see FIG. 1), and the 22 item sinonasal outcome test (SNOT-22). The Sniffin' Sticks olfactory test is a well validated test with a good test-rest reliability (r=0.72) which examines a subject's olfactory threshold (1-butanol), odor discrimination and odor identification (TDI). SNOT-22 is a rhinosinusitis-specific, health-related questionnaire that effectively combines symptoms related to rhinosinusitis and general health.

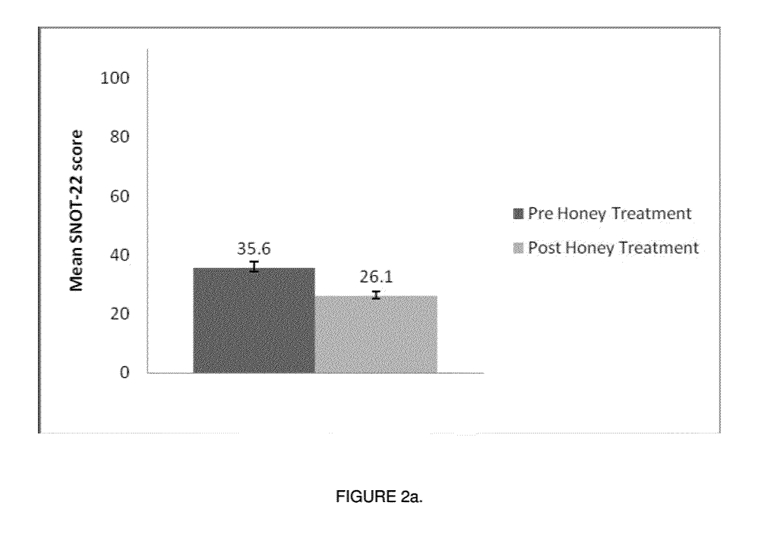

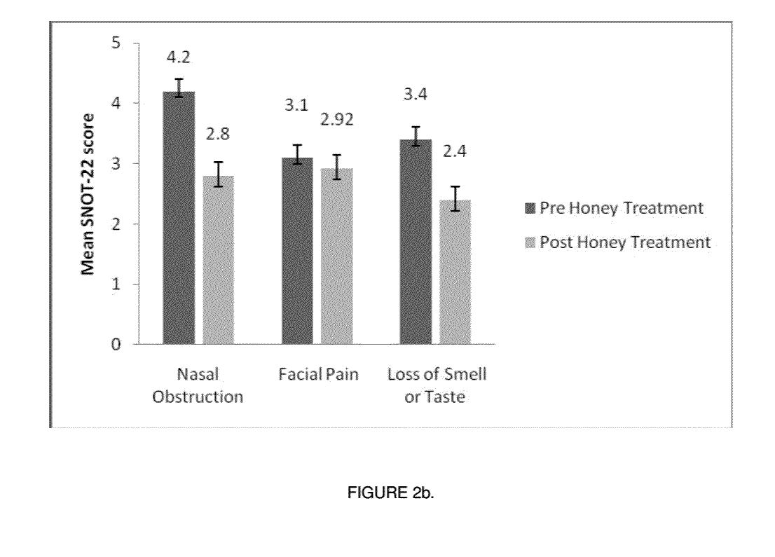

STEP 2: Endoscopic Staging. After completing step 1 above, subjects were examined endoscopically and staged using the newly developed Philpott-Javer Scoring system (see FIG. 2a) which gives a maximum endoscopic score (ES) out of 10 for each sinus cavity bilaterally (possible maximum score of 80).

STEP 3: Treatment with Honey. The honey saline solution used for this study was made by dissolving 10 cc of Wedderspoon® Manuka honey (donated by Wedderspoon Organic®) in 240 mL of pH-balanced sodium chloride and sodium bicarbonate mixture (made by dissolving 1 Sinus Rinse™ mixture packet in 240 mL of distilled water). Patients were given 1 custom 240 mL nasal irrigator, purchased from NeilMed® Pharmaceuticals, Inc., and were instructed to irrigate each of their nostrils with 120 mL of the prepared honey solution, every 24 hours for 30 days. On day 30, last day of the study, step 1 and 2 were repeated for all 10 patients and the three scores (TDI, SNOT-22, ES) were compared to those obtained on Day 1.

Statistical Analysis. For comparing mean scores before and after honey treatment, we calculated the 95% Confidence interval and obtained appropriate error bars.

The following results were obtained from the study:

SNOT-22 Scores. The mean total SNOT-22 score (all 22 items) was 35.6 and 26.1 before and after honey treatment, respectively (see Figure. 2a). This represents a reduction of 26% in symptom scores. More specific results can be obtained by analyzing individual symptoms. In order to compare our results to other published data we selected: (1) nasal obstruction; (2) facial pain; and (3) loss of smell or taste (see Figure. 2b).

Nasal Obstruction. Mean pre-honey treatment score for nasal obstruction was 4.2, which equates to a ‘severe problem’ compared with a post-honey treatment score of 2.8, or a ‘very mild problem’. Seven out of ten patients (70%) reported an improvement of at least 1 point in nasal obstruction.

Facial Pain. Mean pre-honey treatment score was 3.1, mean post-honey treatment score was 2.92. This difference was not statistically significant.

Loss of Smell or Taste. Mean pre-honey treatment score was 3.4 which equates to a ‘moderate problem’, compared to mean a post-honey treatment score of 2.4, or a ‘slight problem’. Six out of ten patients (60%) reported an improvement of at least 1 point in loss of smell or taste.

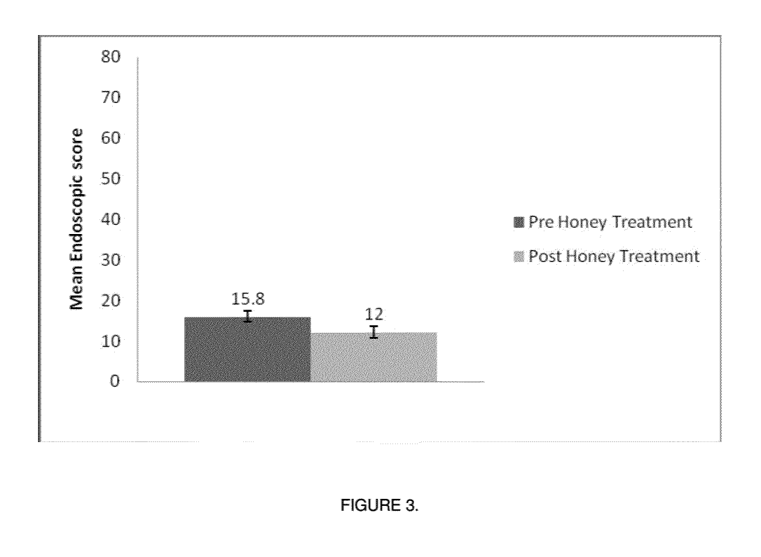

Endoscopic Scores. The mean endoscopic scores using the Philpott-Javer Scoring system were 15.8 and 12 before and after honey treatment, respectively. This difference was not statistically significant (see FIG. 3).

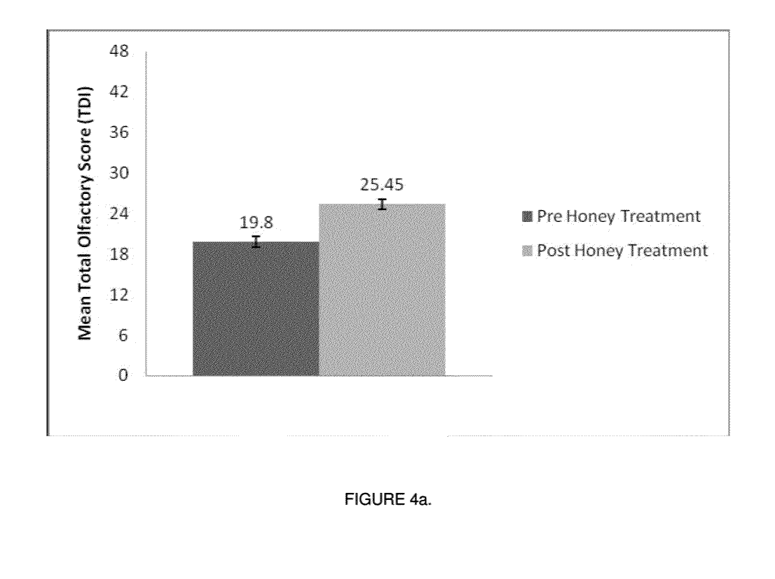

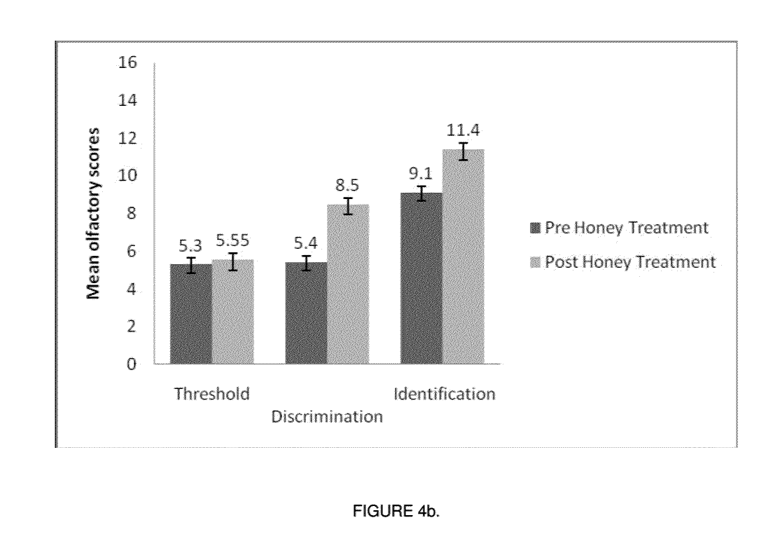

Olfactory Scores. The olfactory score consists of three measures: (1) threshold (T), (2) discrimination (D), and (3) identification (I). The mean total TDI scores were 19.80 and 25.45 before and after honey treatment, respectively (see FIG. 4a). The mean score for T was the only olfactory measure that did not significantly change following honey treatment (5.30 to 5.55) (see FIG. 4b). The mean scores for D and I improved significantly following honey treatment. The greatest improvement was observed for D at 3.1 points followed by 2.3 points for 1 (see FIG. 4b).

Results. After 30 days of sinus irrigation with honey the overall mean scores for olfactory function and SNOT-22 improved significantly, where as endoscopic staging scores remained relatively unchanged. The only olfactory component that did not significantly improve, following treatment with honey, was odor threshold (5.3 to 5.55), which could be due to the use of only a single odorant. Therefore, in spite of no apparent improvement in the sinus cavities, treatment with honey improved the self-reported quality of life scores and the overall olfactory performance scores. Just after 30 days of sinus irrigation with honey, the CF patients' ability to discriminate and identify different odors improved significantly.

### Example 3

### Sinusitis Symptoms in Patients Who Continue to Suffer from Symptoms of Sinusitis after Maximal Topical Medical and Surgical Treatment for AFRS

Materials and Methods. A randomized, single-blinded, prospective study was carried out. Patients included in the study had undergone bilateral functional endoscopic sinus surgery (FESS) and had a definitive diagnosis of allergic fungal sinusitis prior to consideration for the study. Those accepted into the study had to have failed standard medical treatment for at least twelve weeks post-FESS. All patients were 19 years of age or older. Patients allergic to pollen, honey or bees were prohibited from partaking in the study.

The rigid endoscope was used to assess the mucosal inflammation. The mucosa was graded by the staging system used in the clinic where the study was performed, which is based on the old Kupferberg staging. Each sinus cavity (frontal, ethmoid, maxillary and sphenoid) was given a score between zero and nine, with an extra point for the presence of fungal mucin, giving a total potential maximum score of forty out of forty per side of the nose and a total of 80 for both sides of the nose, as set out in Table 2.

TABLE 2

| Sinus cavity | Right | Mucin | Left | Mucin |
|---|---|---|---|---|
| Frontal | 0-9 | 1 | 0-9 | 1 |
| Ethmoid | 0-9 | 1 | 0-9 | 1 |
| Maxillary | 0-9 | 1 | 0-9 | 1 |
| Sphenoid | 0-9 | 1 | 0-9 | 1 |

| | | |
|---|---|---|
| Total | 40 | 40 |

| | |
|---|---|
| Bilateral total | 80 |

| Grading | State of mucosa |
|---|---|
| 0 | No oedema |
| 1-3 | Mucosal oedema (mild/moderate/severe) |
| 4-6 | Polypoid oedema (mild/moderate/severe) |
| 7-9 | Frank polyps (mild/moderate/severe) |

For patients who fitted the inclusion criteria for the study, the senior investigator stepped out of the room and a closed envelope system was use to randomly pick which nostril would receive the honey treatment. Patients acted as their own control. In other words, the patient continued with their current medical management in both nostrils but only one nasal cavity was selected to undergo honey treatment. At this time, microbiology samples were taken from both ethmoid cavities and sent to the laboratory for cultures and sensitivity.

Patients were instructed to spray 2 mL of a 50/50 honey-saline solution using a mucosal atomisation device in their selected nostril once a day at night for thirty days. Patients were seen after thirty days and the senior investigator who was blinded assessed both nostrils and determined which nasal mucosa looked healthier using the endoscopic mucosal grading system. Each patient filled out a validated survey (the SNOT-22 questionnaire) regarding their nasal symptoms during their initial visit and after 4 weeks of treatment.

The honey used in the study is formally known as Medihoney® with active leptospermum honey. The honey was obtained from Derma Sciences, Inc.

To obtain a significant result with a power of 80% (p<0.05), at least 31 patients with AFRS were require to detect a benefit from the treatment. An improvement in the endoscopic mucosal score of five or more points was considered significant. Paired t-tests were used to compare the mucosal and SNOT-22 scores using Stata software.

Results. It was not evident from the results that the 50% solution showed anti-inflammatory and antimicrobial activity. The treatment group had a mean endoscopic mucosal score that was −1.74 units different from the control group (95% CI: −5.02, 1.55), which was not statistically significant (p=0.2901). Further, qualitative analysis of the microbiology results did not reveal consistent trends. Patients grew varying ranges of bacterial flora with no obvious response to the honey treatment, as shown in Table 3 below.

TABLE 3

| | Control | Treatment | Difference | p- |
|---|---|---|---|---|
| | Mean (sd) | Mean (sd) | (95% CI) | value* |
| Mucosal | 18.94 (8.92) | 17.21 (9.90) | −1.74 (−5.02, 1.55) | 0.2901 |
| score | | | | |
| Change in | −0.74 (9.48) | −2.62 (7.27) | −1.88 (−5.04, 1.27) | 0.2334 |
| Mucosal | | | | |
| score | | | | |

*based on a paired t-test.

As for any stated side effects from honey administration, four patients complained of a burning sensation within the sinus cavity. Nonetheless, all four patients were compliant with their honey treatment and were able to continue with the study. On the other hand, one patient documented nausea after spraying the honey and consequently became non-compliant with the study.

### Example 4

### Use of Honey-Saline Solutions in Patients Who Continue to Suffer from Symptoms of Sinusitis after Maximal Topical Medical and Surgical Treatment for AFRS

Materials and Methods. The materials and methods used in Example 3 were used in a prospective study of ten patients, except that the honey-saline solution used by the patients in this study differed. Five patients were instructed to spray 2 mL of a honey-saline solution with 25% v/v of honey: 75% v/v of saline solution, and the other five patients were instructed to spray 2 mL of a honey-saline solution with 15% v/v of honey: 85% v/v of saline solution, in accordance with the instructions set out Example 3. The patients were examined after twelve weeks of treatment.

Results. Both groups of patients were able to tolerate the honey-saline solutions; however, the patients did complain of a burning sensation when using the honey-saline solutions. Endoscopic examination of mucosal inflammation in the treatment groups did not show a statistically significant difference in endoscopic mucosal score over a twelve week period. It was not evident from the results that the 25% solution showed anti-inflammatory and antimicrobial activity.

### Example 5

### Sinusitis Symptoms in Patients Who Continue to Suffer from Symptoms of Sinusitis after Maximal Topical Medical and Surgical Treatment for AFRS

Materials and Methods. The materials and methods used in Example 3 were used in a prospective study of five patients, except that the honey-saline solution used by the patients in this study differed. The patients were instructed to spray 2 mL of a honey-saline solution with 5% v/v of honey: 95% v/v of saline solution, in accordance with the instructions set out Example 3. The patients were examined after twelve weeks of treatment.

Results. The patients were able to tolerate the honey-saline solutions. The endoscopic mucosal scores for the patients improved significantly and the overall mean scores for olfactory function and SNOT-22 improved significantly. The results demonstrate that the 5% solution showed anti-inflammatory and antimicrobial activity.

While preferred embodiments of the present discovery have been shown and described herein, it will be obvious to those skilled in the art that such embodiments are provided by way of example only. Numerous variations, changes, and substitutions will be obvious to those skilled in the art without departing from the discovery. It should be understood that various alternatives to the embodiments of the discovery described herein may be employed in practicing the invention. It is intended that the following claims define the scope of the discovery and that methods and structures within the scope of these claims and their equivalents be covered thereby.

We claim:

1. A composition for treating or alleviating the symptoms of sinusitis, the composition comprising: Manuka honey at a concentration of from about 0.5% v/v to about 7% v/v in an aqueous pH-balanced solution.

2. The composition, according to claim 1, in which the concentration of the Manuka honey is from about 1% v/v to about 7% v/v.

3. The composition, according to claim 1, in which the Manuka honey is present at a concentration from about 3% v/v to about 6% v/v.

4. The composition, according to claim 1, in which the concentration of the Manuka honey is from about 4% v/v to about 5% v/v.

5. The composition, according to claim 1, consisting of 0.5% v/v Manuka honey and 99.5% v/v of the aqueous pH-balanced solution.

6. The composition, according to claim 1, consisting of 5% v/v Manuka honey and 95% of the aqueous pH-balanced solution.

7. The composition, according to claim 1, consisting of 7% v/v Manuka honey and 93% of the aqueous pH-balanced solution.

8. The composition, according to claim 1, consisting essentially of 0.5% v/v Manuka honey and 99.5% of the aqueous pH-balanced solution

9. The composition, according to claim 1, consisting essentially of 5% v/v Manuka honey and 95% of the aqueous pH-balanced solution

10. The composition, according to claim 1, consisting essentially of 7% v/v Manuka honey and 93% of the aqueous pH-balanced solution

11. The composition, according to claim 1, in which the aqueous pH-balanced solution is an aqueous mixture of sodium chloride and sodium bicarbonate.

12. A method of treating, or alleviating the symptoms of, sinusitis in a patient, the method comprising: intranasally administering the composition, according to claim 1, to the patient in need thereof, so as to treat the sinusitis.

13. The method, according to claim 12, in which the composition is administered every 24 hours.

14. The method, according to claim 12, in which the composition is administered every 24 hours for 30 days.

15. The method, according to claim 12, in which the composition is administered irrigate each of their nostrils with about 120 mL of the prepared honey solution, every 24 hours for 30 days

16. The method, according to claim 12, in which the sinusitis is chronic rhinosinusitis.

17. The method, according to claim 12, in which the sinusitis is after functional endoscopic sinus surgery.

18. Method of improving olfactory function in a patient, the method comprising: intranasally administering the composition, according to claim 1, to the patient in need thereof, so as to improve the olfactory function.

19. The method, according to claim 18, in which the composition is administered every 24 hours.

20. The method, according to claim 18, in which the composition is administered every 24 hours for 30 days.

21. The method, according to claim 18, in which the composition is administered irrigate each of their nostrils with about 120 mL of the prepared honey solution, every 24 hours for 30 days
